(19) United States
(12) Patent Application Publication
(10) Pub. No.: US 2024/0198074 A1
(43) Pub. Date: Jun. 20, 2024

(54) MEDICAL ADAPTER GRIPPING TOOL

(71) Applicant: The Children’s Mercy Hospital, Kansas City, MO (US)

(72) Inventor: David Keeler, Kansas City, MO (US)

(21) Appl. No.: 18/555,284

(22) PCT Filed: Apr. 15, 2022
(86) PCT No.: PCT/US2022/025044

§ 371 Date: Oct. 13, 2023

Related U.S. Application Data
(60) Provisional application No. 63/175,180, filed on Apr. 15, 2021.

(51) Int. Cl. A61M 39/10; A61M 25/00; B25B 7/04; B25B 7/08

(52) U.S. Cl. CPC A61M 39/10; A61M 25/0097; B25B 7/04; B25B 7/08; A61M 2039/1077; A61M 2209/04

(57) ABSTRACT

A medical adapter gripping tool is operable to removably hold a catheter element and includes a pair of elongated tool levers. Each tool lever includes a longitudinally extending lever body and presents opposite lever ends. The tool levers are pivotally attached relative to one another at a pivot joint that permits the tool levers to swing relative to each other about a lateral pivot axis. The tool levers are relatively swingable into and out of a closed position to grip the catheter element.

### CROSS-REFERENCE TO RELATED APPLICATIONS

The present application claims the priority benefit of U.S. Provisional Patent Application Ser. No. 63/175,180, filed Apr. 15, 2021, entitled MEDICAL ADAPTER GRIPPING TOOL, incorporated by reference in its entirety herein.

### BACKGROUND

### Field

The present invention relates generally to medical equipment. More specifically, embodiments of the present invention concern a gripping tool for removably holding a hub, adapter, Luer-Lok™, ENFit® connector, needleless connector, and/or other connectors used with catheters or other medical devices.

### Discussion of Related Art

A catheter is a thin tube made from medical grade materials serving a broad range of functions. Catheters are medical devices that can be inserted in the body to treat diseases or perform a surgical procedure. By modifying the material or adjusting the way catheters are manufactured, it is possible to tailor catheters for cardiovascular, urological, gastrointestinal, neurovascular, and ophthalmic applications. Various types of catheters, such as a central venous catheter (i.e., a “central line”), are commonly used to provide intravenous (IV) access to a patient. Known catheters include a catheter lumen and a catheter hub for removable attachment to a catheter adapter. In the usual manner, the hub and adapter include a fractional-turn quick connector (such as a Luer-Lok™) for selective attachment and detachment of the adapter relative to the hub. This connection serves to keep the IV system closed to the outside environment.

However, conventional IV catheters and adapters have various deficiencies. For instance, prior art catheter hubs and adapters are known to become “stuck” or “fused” to each other when connected, such that disconnection becomes difficult. A stuck hub-adapter connection may be associated with a variety of factors. For instance, the catheter may be used to deliver fluids, such as a total parenteral nutrition (TPN) fluid, to an infant patient, and the fluids may produce a sticky residue that restricts disconnection of the hub and adapter. Conventional hub-adapter connections are also prone to accidental over-tightening of the connection, which may cause difficulty in disconnecting the hub and adapter. Because the hub and adapter are relatively small compared to the size of an adult's hands, it can be difficult to manually disconnect the hub and adapter with their hands. Furthermore, common medical tools, such as gloves and clamps, are ill-suited to firmly grasping the hub or adapter for disconnection. When grasping the hub or adapter with a clamp, the clamp may easily damage the catheter, such that catheter removal and replacement is required. Catheter replacement typically requires a surgical procedure and incurs additional treatment costs. A risk of infection may also result from catheter damage or contamination of the catheter system.

### SUMMARY

The following brief summary is provided to indicate the nature of the subject matter disclosed herein. While certain aspects of the present invention are described below, the summary is not intended to limit the scope of the present invention.

Embodiments of the present invention provide a medical adapter gripping tool that does not suffer from the problems and limitations of prior art devices, including those set forth above.

A first aspect of the present invention concerns a medical adapter gripping tool operable to removably hold a catheter element. The medical adapter gripping tool broadly includes a pair of elongated tool levers. Each tool lever includes a longitudinally extending lever body and presents opposite lever ends. The tool levers are pivotally attached relative to one another at a pivot joint that permits the tool levers to swing relative to each other about a lateral pivot axis, with the tool levers being relatively swingable into and out of a closed position to grip the catheter element. Each tool lever defines a jaw located between the lever ends. The jaws are opposed and swingable toward and away from each other, with the jaws cooperatively engaging the catheter element in the closed position. The respective tool lever includes a gripping element supported by and cooperating with the respective lever body to form a corresponding jaw, with the gripping element presenting an exposed gripping surface. The gripping element comprises a resilient gripping element material, with the gripping element operable to resiliently flex for conforming with part of the catheter element and restricting the gripping force applied by the jaws when the catheter element is held by the jaws in the closed position.

A second aspect of the present invention concerns a medical adapter gripping tool operable to removably hold a catheter element. The medical adapter gripping tool broadly includes a pair of elongated tool levers. Each tool lever includes a longitudinally extending lever body and presents opposite lever ends. The tool levers are pivotally attached relative to one another at a pivot joint that permits the tool levers to swing relative to each other about a lateral pivot axis, with the tool levers being relatively swingable into and out of a closed position to grip the catheter element. Each tool lever defines a jaw located between the lever ends. The jaws are opposed and swingable toward and away from each other, with the jaws cooperatively engaging the catheter element in the closed position. At least one of the tool levers includes proximal and distal lever sections and a resilient hinge that joins the lever sections and permits relative flexing between the lever sections about a lateral flexing axis to restrict the force applied by the jaws when the catheter element is held by the jaws in the closed position.

A third aspects of the present invention concerns a method of removably gripping a catheter element with a medical adapter gripping tool. The method broadly includes the steps of having the catheter element positioned between the jaws of pivotally-attached tool levers while the tool levers are opened; and having the tool levers shifted relative to each other to close the jaws against the catheter element, with a gripping element of at least one jaw resiliently flexing to conform with part of the catheter element and restrict the gripping force applied by the jaws when the catheter element is held by the jaws in the closed position.

This summary is provided to introduce a selection of concepts in a simplified form that are further described below in the detailed description. This summary is not intended to identify key features or essential features of the claimed subject matter, nor is it intended to be used to limit the scope of the claimed subject matter. Other aspects and advantages of the present invention will be apparent from the following detailed description of the embodiments and the accompanying drawing figures.

### BRIEF DESCRIPTION OF THE DRAWING FIGURES

Preferred embodiments of the invention are described in detail below with reference to the attached drawing figures, wherein:

FIG. 1 is an upper perspective view of a medical adapter gripping tool constructed in accordance with a preferred embodiment of the present invention, said gripping tool including a pair of elongated tool levers with opposed flexible gripping elements, with the tool levers being opened to receive a catheter hub between the gripping elements;

FIG. 2 is an upper perspective view of the medical adapter gripping tool similar to FIG. 1, but showing the tool levers closed so that the gripping elements grip the catheter hub;

FIG. 3 is a lower perspective view of the medical adapter gripping tool shown in FIGS. 1 and 2, showing the tool levers pivotally attached by a torsion spring;

FIG. 4 is an upper perspective view of the medical adapter gripping tool similar to FIG. 3, but taken from the opposite side to depict the gripping elements;

FIG. 5 is a fragmentary perspective view of the medical adapter gripping tool shown in FIGS. 1-4, showing portions of the tool levers broken away to depict prongs of the torsion spring inserted into slotted openings presented by the tool levers;

FIG. 6 is a perspective view of the medical adapter gripping tool shown in FIGS. 1-5, showing the tool levers in the closed position;

FIG. 7 is a side elevational view of the medical adapter gripping tool shown in FIGS. 1-6, showing a slotted opening defined between the gripping elements in the closed position;

FIG. 8 is a cross-sectional view of the medical adapter gripping tool taken along line 8-8 in FIG. 7;

FIG. 9 is an upper perspective view of a metical adapter gripping tool constructed in accordance with a second preferred embodiment of the present invention, said gripping tool including a pair of elongated tool levers with opposed flexible gripping elements, with the tool levers being opened to receive a catheter hub between the gripping elements;

FIG. 10 is an upper perspective view of the medical adapter gripping tool similar to FIG. 9, but showing the tool levers opened to a greater degree to permit removal of one gripping element from a corresponding jaw frame;

FIG. 11 is an upper perspective view of the medical adapter gripping tool similar to FIG. 10, but with the gripping tool being cross sectioned along the length thereof, and showing both gripping elements located in the respective jaw frames; and

FIG. 12 is an elevation view of the medical adapter gripping tool shown in FIGS. 9-11, showing the tool levers in the open position.

The drawing figures do not limit the present invention to the specific embodiments disclosed and described herein. The drawings are not necessarily to scale, emphasis instead being placed upon clearly illustrating the principles of the preferred embodiment.

### DETAILED DESCRIPTION OF THE PREFERRED EMBODIMENTS

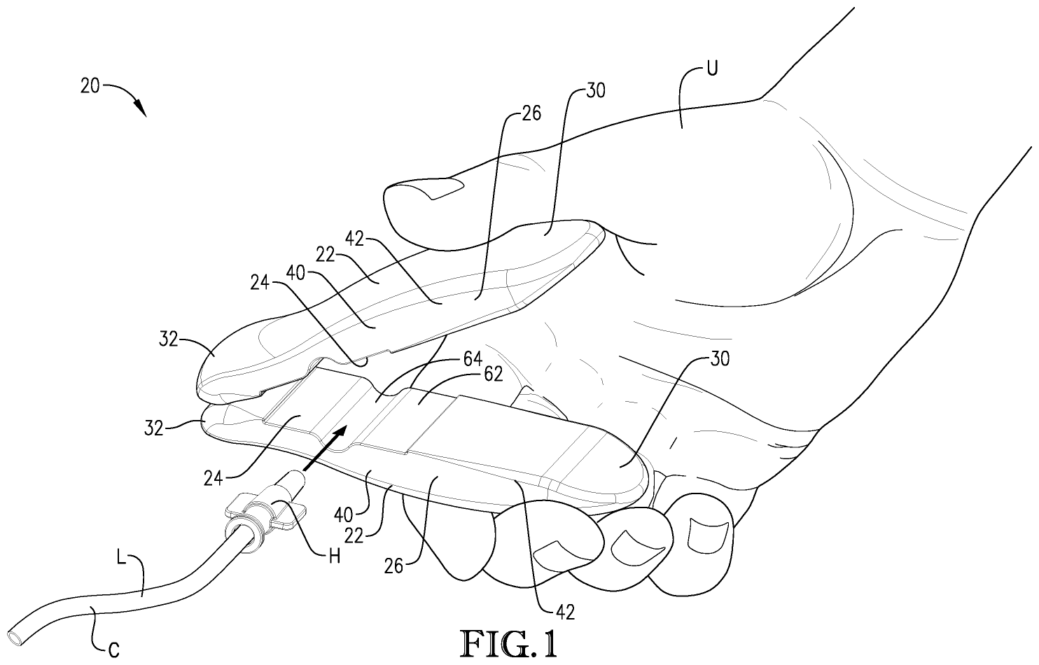

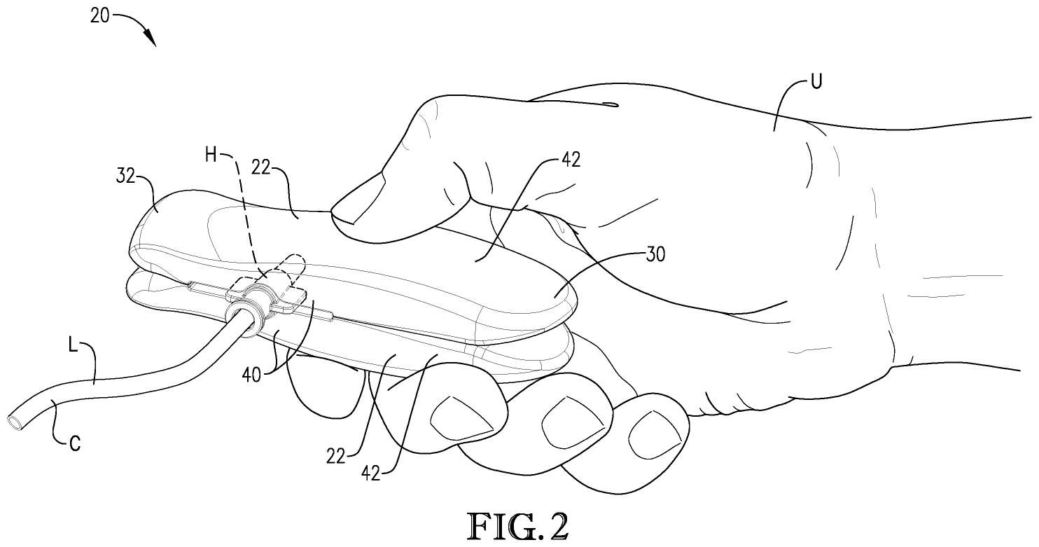

Turning initially to FIGS. 1 and 2, a medical adapter gripping tool 20 is configured to removably hold a catheter element, particularly when a catheter adapter (not shown) is being attached to or detached from a catheter hub H. For instance, as will be described, the gripping tool 20 is configured to removably grip the catheter hub H and hold the catheter hub H during attachment and detachment of the hub-adapter connection.

It will be appreciated that the gripping tool 20 is configured to grip the catheter hub H by being manually held in clamping engagement with the catheter hub H. For at least some aspects of the present invention, the gripping tool 20 may include a releasable mechanism to hold itself in clamping engagement with a respective catheter element. The preferred gripping tool 20 includes a pair of elongated tool levers 22, which have corresponding flexible gripping elements 24, as will be discussed. The depicted gripping elements 24 are integrally formed as part of the tool levers 22. However, as will be described further, one or more of the gripping elements may comprise inserts that are received by a frame of the lever (e.g., where the lever body and gripping element are formed of different materials).

The illustrated central venous catheter C (also referred to as a “central line”) is configured to provide intravenous access to a patient. The catheter C includes a catheter lumen L and a catheter hub H for removable attachment to a catheter adapter (not shown). It is also within the scope of the present invention to use the gripping tool with other types of medical catheters, such as alternative intravenous (IV) catheters (e.g., a peripheral venous catheter, a PICC line, etc.).

Tool levers 22 are configured to cooperatively grip and hold the catheter hub H for attachment and detachment of the hub-adapter connection. Tool levers 22 each include a longitudinally extending lever body 26 and the flexible gripping element 24 (see FIG. 1).

Turning to FIGS. 2-8, each tool lever 22 includes proximal and distal lever sections 30,32 and a resilient hinge 34 that joins the lever sections. As will be discussed, the resilient hinge 34 permits relative flexing between the lever sections 30,32 to restrict the clamping force applied by the jaws when the gripping tool 20 is in a closed position.

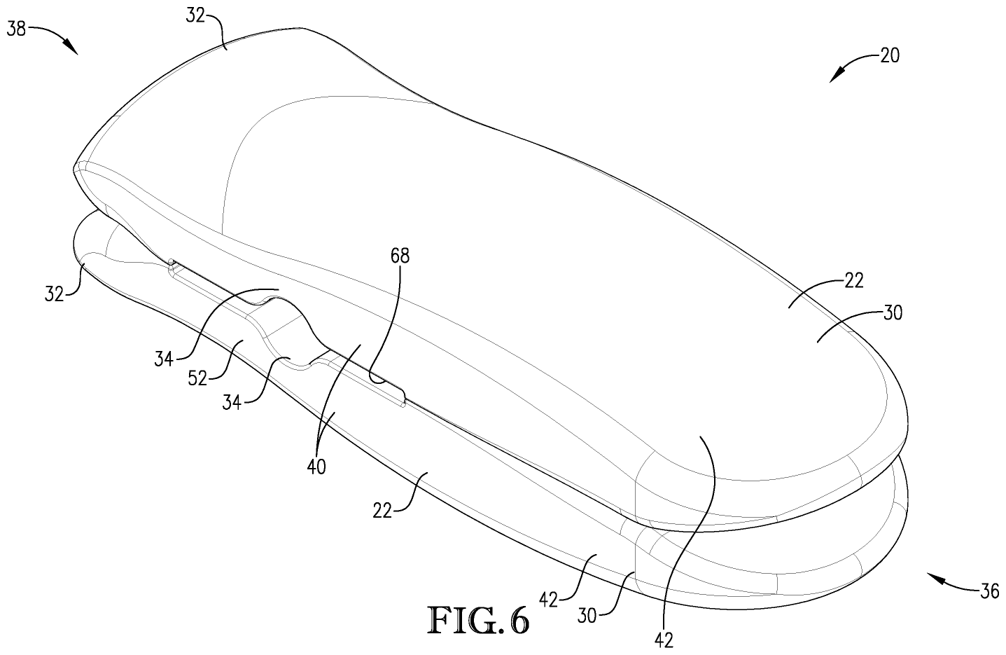

Each tool lever 22 also presents proximal and distal lever ends 36,38 and includes a jaw 40 located between the lever ends 36,38 (see FIG. 6). The terms “proximal” and “distal” refer to locations along the lever body 26 relative to a tool handle 42 of the tool lever 22 (see FIG. 6). The tool handles 42 are manually grasped by the hand U of a user to close the gripping tool 20 (see FIGS. 1 and 2).

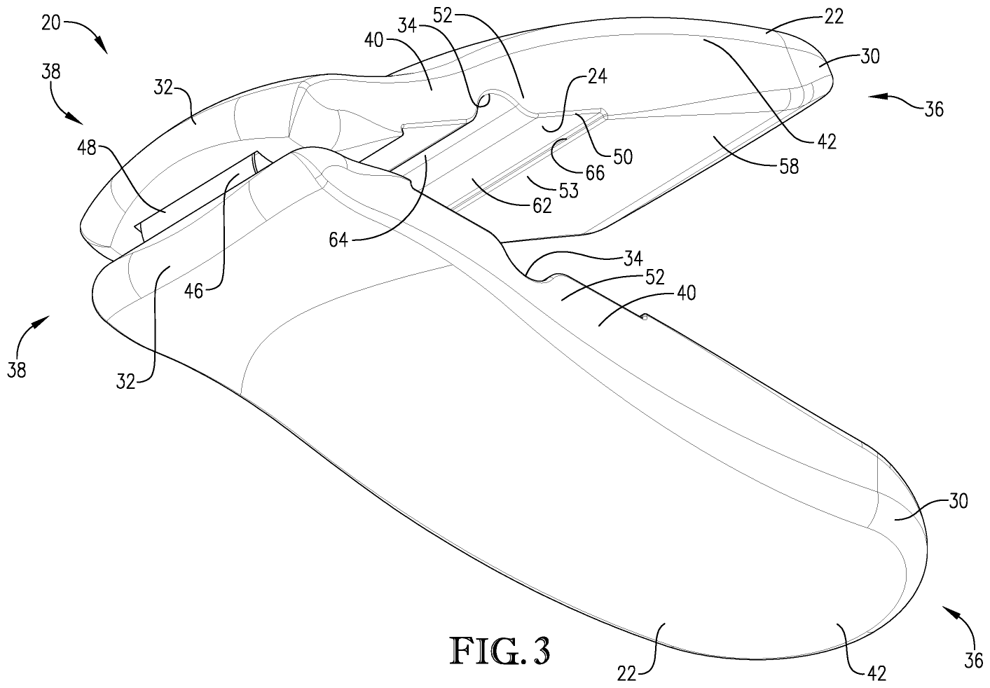

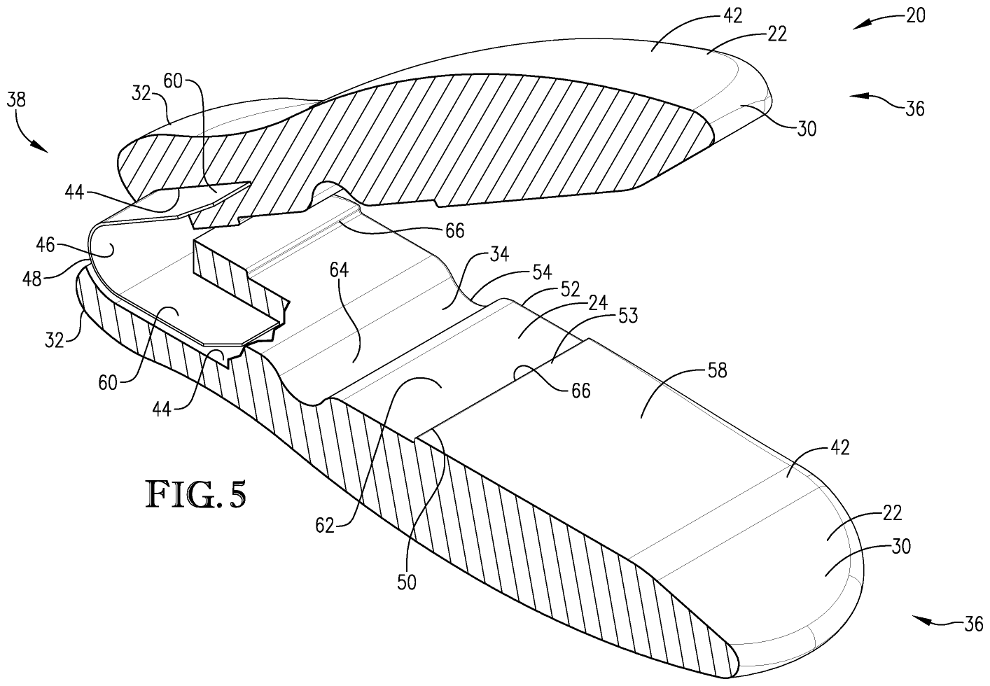

Each lever body 26 is unitary and extends continuously between the proximal and distal lever ends 36,38. The distal lever ends 38 preferably comprise connection elements that each present a slotted opening 44 (see FIG. 5). As will be explained, the connection elements are attached to one another by a torsion spring 46 to form a pivot joint 48 (see FIGS. 3 and 5), although the pivot joint may be alternatively constructed. The lever body 26 includes an outer jaw margin 50 that extends around the integrally-formed gripping element 24. Preferably, the outer jaw margin 50 surrounds and supports the gripping element 24.

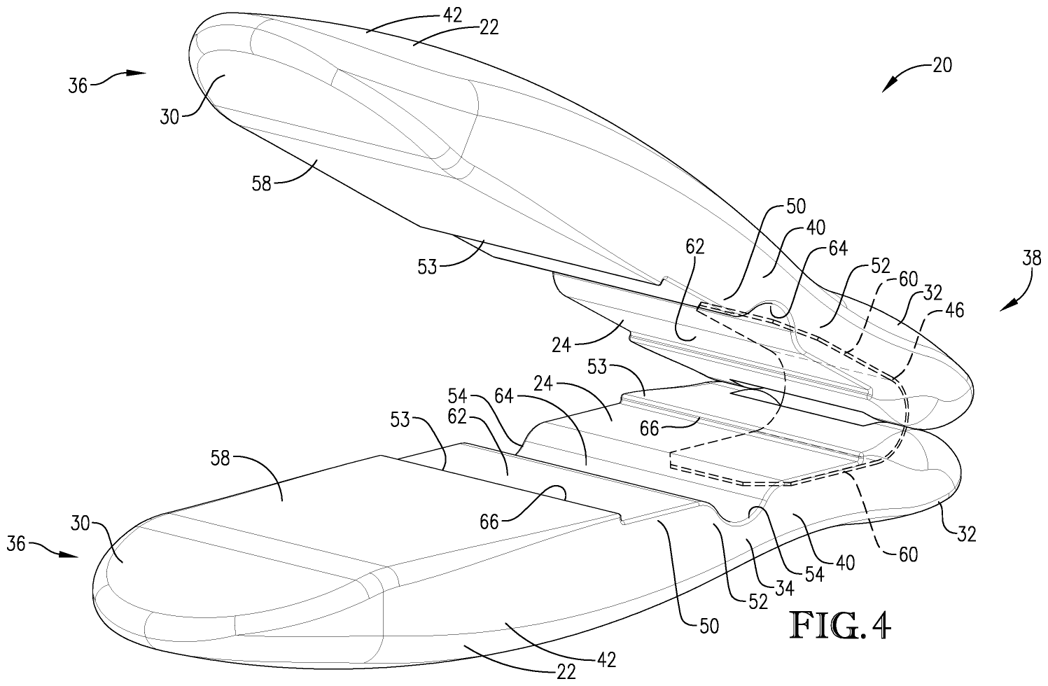

The outer jaw margin 50 of the illustrated embodiment includes a pair of notched side sections 52 that extend longitudinally alongside the gripping element 24 and opposite end sections 53 (see FIGS. 4 and 8). In preferred embodiments, the outer jaw margin 50 is integrally formed with the respective gripping element 24. However, for at least certain aspects of the present invention, the gripping element may be attached relative to the outer jaw margin. For instance, embodiments of a gripping tool may have a jaw with side wall and end walls that cooperatively present a socket to receive a gripping element in the form of an insert. Additional preferred details of a gripping tool with a gripping element insert are described in another embodiment below.

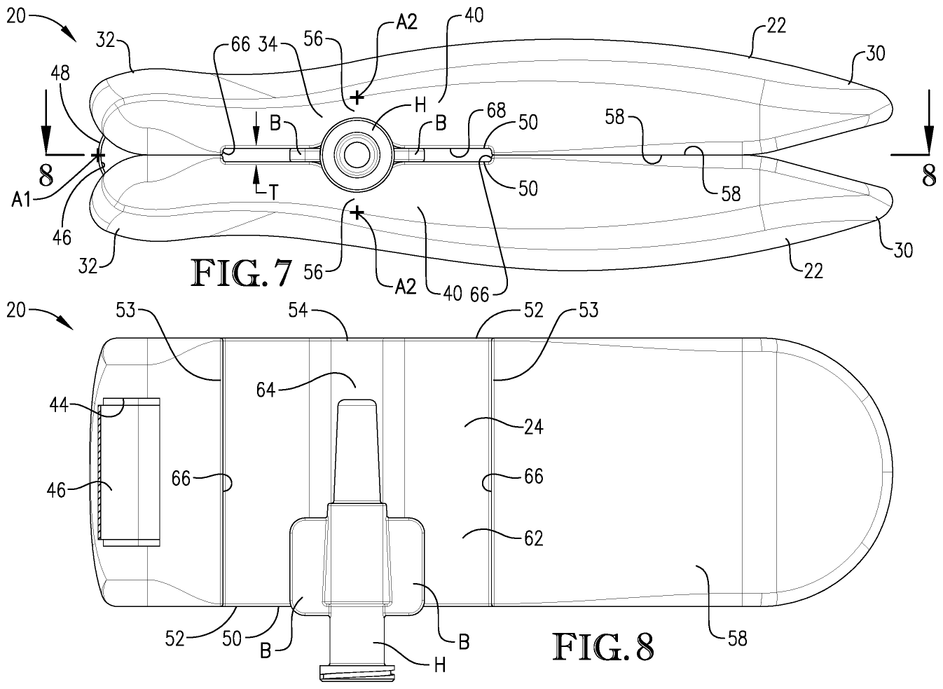

Side sections 52 of the outer jaw margin present notched openings 54 that are longitudinally aligned with one another and with a channel presented by the gripping element 24 (see FIG. 4). Preferably, the notched openings 54 are associated with a living hinge 56 provided by the outer jaw margin 50 (see FIG. 7). As will be described, the living hinge 56 is preferably provided as part of the resilient hinge 34 to permit relative flexing between the lever sections 30,32.

Although each lever body 26 preferably includes the living hinge 56, at least one lever body may be provided with an alternative hinge to facilitate relative flexing between the lever sections. For instance, consistent with at least certain aspects of the present invention, the jaw may include a hinge with discrete hinge elements (e.g., to form a pinned hinge joint) instead of a living hinge.

While the jaw 40 preferably defines the hinge 34 separating the lever sections 30,32, for at least some aspects of the present invention, the resilient hinge may be provided by part of the lever body other than the jaw. For instance, the hinge separating the lever sections may be located between the proximal lever end and the jaw.

In preferred embodiments, each lever body 26 includes a lever material that comprises a synthetic resin material. The lever material preferably material permits the lever body (particularly the living hinge) to flex and comprises substantially the same material as the gripping element 24. However, alternative tool embodiments may include a lever material that is relatively rigid when compared to the gripping element material of the respective gripping element. Exemplary embodiments of such a tool are described below in the second preferred embodiment.

As will be explained, the flexibility associated with the lever body 26 enables the tool lever 22 to bend slightly to accommodate a catheter hub H, while still reaching a “hard stop point” when planar stop surfaces 58 associated with the handles 42 contact each other (see FIG. 7).

The lever material may include one or more polymer materials, such as a high density polyethylene (HDPE), nylon, ABS, polyethylene terephthalate (PET), polyvinyl chloride (PVC), and/or polypropylene. Additionally or alternatively, the lever material may include renewable plastics such as polylactic acid or polybutylene succinate. Preferably, the lever material has a Shore D durometer that ranges from about 70 to about 80.

The tool levers 22 of the illustrated embodiment are preferably substantially identical to one another. However, it is within the ambit of the present invention for the tool levers to have different configurations. For example, in alternative gripping tool embodiments, only one of the tool levers may include a flexible gripping element.

In the depicted embodiment, tool levers 22 are pivotally attached relative to one another at the pivot joint 48 (see FIG. 3). Specifically, distal ends 38 of the illustrated tool levers 22 are pivotally attached to each other via the torsion spring 46, which permits the tool levers 22 to swing relative to each other about a lateral pivot axis A1 (see FIG. 7). In particular, the distal ends 38 of tool levers 22 include connection elements that define slotted openings 44 to receive prongs 60 of the spring 46 (see FIG. 5). However, the pivot joint for swingably connecting the tool levers may be cooperatively formed by alternative pivot connectors. An alternative pivot joint may include a structure other than the disclosed torsion spring (such as a pin or threaded fastener) for joining the tool connectors. In alternative embodiments, the pivot joint may be provided by a living hinge. The tool levers are relatively swingable into and out of a closed position (see FIGS. 2 and 7) to grip the catheter hub (or another part of the catheter).

Tool handles 42 of the tool levers 22 extend adjacent the proximal lever ends 36 and are configured to be grasped by a user to swing the tool levers 22 between the closed position (see FIG. 2) and an open position (see FIGS. 1 and 3). The spring 46 is preferably configured so that the tool levers 22 are normally located in the open position. Correspondingly, when the levers 22 are located in the closed position, the spring 46 urges the levers 22 to move away from each other and toward the open position. For at least certain aspects of the present invention, alternative embodiments of the tool may have a spring that normally locates the tool levers in the closed position.

The tool handles 42 preferably present opposed stop surfaces 58 (see FIGS. 2 and 3) that engage one another in the closed position to restrict the gripping force applied by the jaws 40. The depicted stop surfaces 58 preferably have a planar shape and are generally in “flush” face-to-face engagement with one another in the closed position. However, one or both stop surfaces may include an alternative surface shape (e.g., with convex and/or concave surfaces) to provide suitable abutting engagement with each other.

The gripping tool 20 is configured so that the jaws 40 are located longitudinally between the pivot joint 48 and the handles 42. For at least some aspects of the present invention, the tool handles may be alternatively positioned relative to the jaws and the pivot joint. For instance, alternative tool levers may extend longitudinally on both sides of the pivot joint so that the tool handles are both on one side of the pivot joint and the jaws are both on the other side of the pivot joint.

Each flexible gripping element 24 is supported by and cooperates with the respective lever body 26 to form a corresponding jaw 40 of the gripping tool 20. The jaws 40 are opposed and swingable toward and away from each other. Jaws 40 of the gripping tool 20 cooperatively engage the catheter hub H in the closed position. As will be explained, the outer jaw margin 50 of the lever body 26 is configured to resiliently flex the gripping element 24 when the gripping tool 20 is closed. The gripping element 24 has a unitary form and presents an exposed gripping surface 62 (see FIGS. 4 and 8). Each gripping surface 62 defines a lateral channel 64 to receive part of the catheter C, such as the catheter hub H (see FIGS. 4 and 8).

In the illustrated embodiment, gripping surface 62 is preferably recessed relative to the adjacent end sections 53 and the stop surface 58 so that shoulders 66 are formed along opposite ends of the gripping surface 62 (see FIGS. 5-8). When the gripping tool 20 is closed, the opposed gripping surfaces 62 are preferably spaced apart and cooperatively define a slotted opening 68 to receive tabs B of the catheter hub H (see FIGS. 5-8). The slotted opening 68 presents a thickness dimension T that generally corresponds to a thickness dimension of the tabs B (see FIG. 7).

For at least certain aspects of the present invention, the slotted opening may have a smaller thickness dimension than the tab thickness of the catheter hub. It is also within the scope of certain aspects of the present invention for tool embodiments to be devoid of a slotted opening between the gripping surfaces when the tool is closed. For instance, gripping elements of the tool may include a relatively flexible elastomeric material that flexes to accommodate a catheter hub when the tool is closed.

As noted above, the depicted gripping element 24 is integrally formed with the lever body 26. However, in alternative embodiments, the gripping element and lever body may be formed of different materials but fixed relative to each other (e.g., where a relatively flexible gripping element is molded or adhered to a relatively rigid lever body). Although the lever body 26 and gripping element 24 are formed of the same or similar material, it will also be understood that an alternative gripping element and lever body may be formed of different materials.

It is also within the scope of certain aspects of the present invention for the gripping element to comprise a flexible insert that is supported by the lever body. For instance, an alternative gripping element may be inserted into the socket and attached therein (the second preferred embodiment described below). Alternative gripping elements may be removably fastened to the lever body with one or more fasteners or otherwise removably supported by the lever body.

It is also within the ambit of the present invention for alternative gripping element embodiments to be fixed relative to the lever body (e.g., where the gripping element is adhered or molded onto the lever body) without being integrally formed with the lever body.

The gripping element is operable to resiliently flex for conforming with part of the catheter element and for restricting the gripping force applied by the jaws when the catheter element is held by the jaws in the closed position. However, it is within the scope of certain aspects of the present invention for the tool to grip the catheter in the closed position without any flexing of the jaws and/or gripping elements.

The gripping element material may include one or more polymer materials, such as a high density polyethylene (HDPE), nylon, ABS, polyethylene terephthalate (PET), polyvinyl chloride (PVC), and/or polypropylene. Additionally or alternatively, the gripping element material may include renewable plastics such as polylactic acid or polybutylene succinate. Preferably, the gripping element material has a Shore D durometer that ranges from about 70 to about 80.

In alternative embodiments, the gripping element may comprise a resilient gripping element material that is relatively more flexible than a material of the respective lever body (see the second preferred embodiment described below).

It is within the scope of the present invention for at least one of the gripping elements to be alternatively configured (e.g., for being supported by the lever body). For instance, at least one of the gripping elements may be alternatively shaped for removable engagement with the lever body. It will also be understood that the gripping surface may be alternatively shaped for alternative gripping engagement with a structure (e.g., a catheter element) to be grasped by the gripping tool.

For at least some aspects of the present invention, a tool lever of an alternative gripping tool may not include a flexible gripping element (e.g., where only one of the tool levers includes a flexible gripping element). For certain aspects of the present invention, both tool levers may be devoid of a flexible gripping element supported by the lever body. In alternative embodiments where one or both tool levers do not include a flexible gripping element, it will be understood that the lever body may include a jaw presenting a gripping surface. Furthermore, such an alternative jaw may comprise a resilient hinge.

As noted above, the resilient hinge 34 of each tool lever 22 permits relative flexing between the proximal and distal lever sections 30,32. In particular, the resilient hinge 34 joins the lever sections 30,32 and permits relative flexing about a lateral flexing axis A2 (see FIG. 7) to restrict the clamping force applied by the jaws 40 when the gripping tool 20 is in the closed position. In the depicted embodiment, the outer jaw margin 50 and the gripping element 24 cooperatively provide the resilient hinge 34.

Each resilient hinge 34 is configured to be shifted between a relaxed condition (see FIG. 1) and a flexed condition. When shifting from the relaxed condition to the flexed condition, one or both of the proximal lever sections 30 of the tool levers 22 are pivoted about the hinges 34 toward each other (see FIGS. 2, 6 and 7). When shifting the depicted lever sections 30 toward the flexed condition, the side sections 52 and end sections 53 of the jaw 40 are pivoted about the corresponding hinge 34 toward each other. When shifting from the flexed condition to the relaxed condition, the proximal lever sections 30 of the tool levers 22 are pivoted about the hinges 34 away from each other. It is also within the scope of certain aspects of the present invention for the tool to grip the catheter in the closed position without any flexing of the jaws and/or gripping elements into or out of the flexed condition.

Again, the lever body 26 includes an outer jaw margin 50 that extends at least partly around the gripping element 24. In the illustrated embodiment, the resilient hinge 34 includes the living hinge 56 provided by the outer jaw margin 50 (see FIG. 7). More particularly, the side sections 52 of the outer jaw margin 50 at least partly define the living hinge 56.

The flexibility associated with the lever body 26, particularly the living hinge 56 of the outer jaw margin 50, permits the tool lever 22 to bend slightly to accommodate the clamped catheter element, while reaching a “hard stop point” when the stop surfaces 58 adjacent the proximal ends 36 contact each other (see FIGS. 2 and 7). As a result, the flexible lever body 26 is configured to restrict the clamping force applied by the jaws 40 when the gripping tool 20 is in the closed position. The living hinge 56 preferably urges the tool lever 22 to return from the flexed condition toward the relaxed condition.

Again, the gripping element 24 cooperates with the outer jaw margin 50 to provide the resilient hinge 34. The end sections 53 of the outer jaw margin 50 are located on opposite ends of the gripping element 24. As the tool lever 22 is shifted toward the flexed condition, the end sections 53 cooperatively resiliently flex the gripping element 24 so that the gripping element 24 provides a spring of the resilient hinge 34. Preferably, the flexed gripping element 24 urges the tool lever 22 to return from the flexed condition toward the relaxed condition. Thus, in the illustrated embodiment, the gripping element 24 and outer jaw margin 50 cooperate to urge the tool lever 22 out of the flexed condition toward the relaxed condition.

In use, the gripping tool 20 is configured to grip a catheter hub H by manually closing the tool levers 22 to hold the jaws 40 in clamping engagement with the catheter hub H. The flexibility of the lever body 26 and gripping element 24 enables bending of the tool levers 22 to accommodate the catheter hub H. At the same time, flexing of the tool levers 22 restricts the gripping tool 20 from applying an excessive gripping force to the catheter hub H (e.g., such that the catheter hub H or another part of the catheter C is damaged).

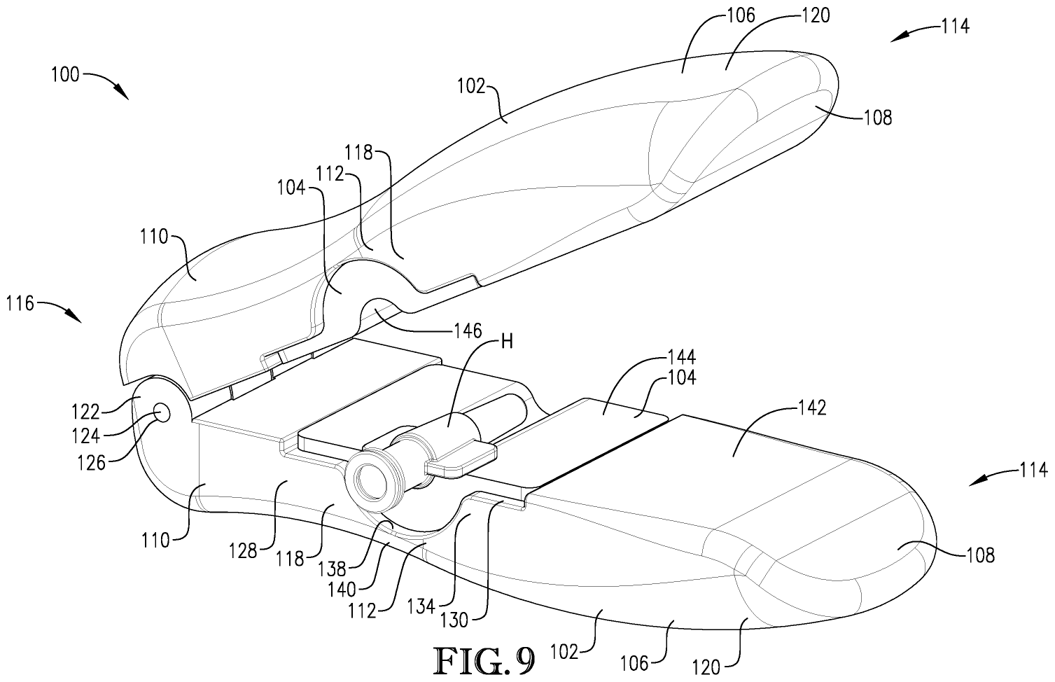

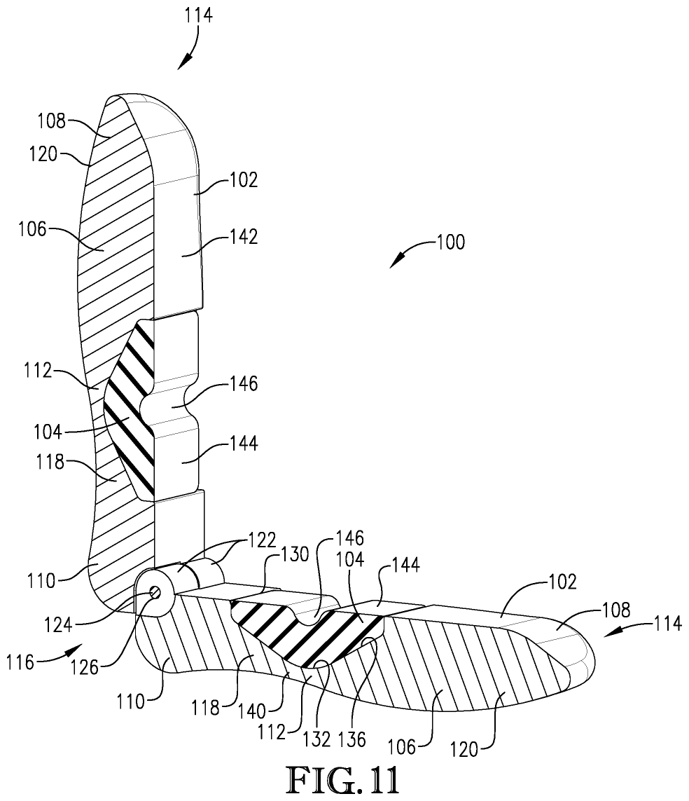

Turning to FIGS. 9-12, a second preferred medical adapter gripping tool 100 of the present invention is disclosed. For the sake of brevity, the remaining description will focus primarily on the differences of this second preferred embodiment from the preferred gripping tool 20 described above.

It will be appreciated that the gripping tool 100 is configured to grip the catheter hub H by being manually held in clamping engagement with the catheter hub H. Embodiments of the gripping tool include a pair of elongated tool levers 102, which have corresponding flexible gripping elements 104, as will be discussed. The depicted gripping elements 104 preferably comprise elastomeric inserts.

Tool levers 102 each include a longitudinally extending lever body 106 and the flexible gripping element 104. Each tool lever 102 includes proximal and distal lever sections 108, 110 and a resilient hinge 112 that joins the lever sections 108,110. As with the previous embodiment, the resilient hinge 112 permits relative flexing between the lever sections 108,110 to restrict the clamping force applied by the jaws when the gripping tool 100 is in a closed position.

Each tool lever 102 also presents proximal and distal lever ends 114,116 and includes a jaw 118 located between the lever ends 114,116. Tool handles 120 are manually grasped by a user to open and close the gripping tool 100.

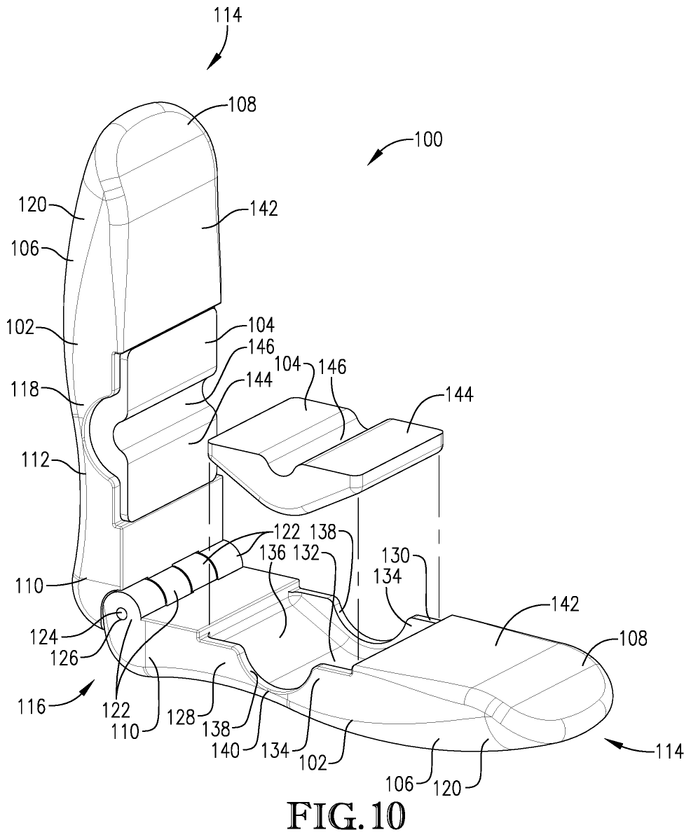

Each lever body 106 is unitary and extends continuously between the proximal and distal lever ends 114,116. The distal lever ends 116 preferably include pivot connectors 122. As will be explained, the pivot connectors 122 are attached to one another by a pin 124 to form a pivot joint 126, although the pivot joint may be alternatively constructed. The lever body 106 includes a jaw frame 128 that extends at least partly around the gripping element 104 and defines an outer jaw margin 130 (see FIG. 10). Preferably, the jaw frame 128 surrounds and at least partly defines a socket 132 to receive and support the gripping element 104 (see FIG. 10).

The jaw frame 128 of the illustrated embodiment includes a pair of notched side walls 134 and end walls 136 that extend alongside the gripping element 104 (see FIG. 10). The side walls 134 and end walls 136 cooperatively present the socket 132 (see FIG. 10). Side walls 134 of the jaw frame 128 present notched openings 138 that are longitudinally aligned with one another (see FIG. 10). Preferably, the notched openings 138 are associated with a living hinge 140 provided by the jaw frame 128 (see FIG. 10). As will be described, the living hinge 140 is preferably provided as part of the resilient hinge 112 to permit relative flexing between the lever sections 108,110.

Although each lever body 106 preferably includes the living hinge 140, at least one lever body may be provided with an alternative hinge to facilitate relative flexing between the lever sections. Again, consistent with at least certain aspects of the present invention, the jaw may include a hinge with discrete hinge elements (e.g., to form a pinned hinge joint) instead of a living hinge.

While the jaw 118 preferably defines the hinge 140 separating the lever sections 108, 110, for at least some aspects of the present invention, a hinge may be provided by part of the lever body other than the jaw. For instance, a hinge separating the lever sections may be located between the proximal lever end and the jaw.

In preferred embodiments, each lever body 106 includes a lever material that comprises a synthetic resin material. The lever material is preferably relatively rigid when compared to the gripping element material of the respective gripping element, although the lever material permits the lever body 106 (particularly the living hinge 140) to flex. As will be explained, the flexibility associated with the lever body 106 enables the tool lever to bend slightly to accommodate a catheter element, while still reaching a “hard stop point” when stop surfaces 142 associated with the tool handles 120 contact each other.

Similar to the previous embodiment, the lever material may include one or more polymer materials, such as a high density polyethylene (HDPE), nylon, ABS, polyethylene terephthalate (PET), polyvinyl chloride (PVC), and/or polypropylene. Additionally or alternatively, the lever material may include renewable plastics such as polylactic acid or polybutylene succinate. Preferably, the lever material has a Shore D durometer that ranges from about 70 to about 80.

The tool levers 102 of the illustrated embodiment are preferably substantially identical to one another. However, it is within the ambit of the present invention for the tool levers to have different configurations. For example, in alternative gripping tool embodiments, only one of the tool levers may include a flexible gripping element.

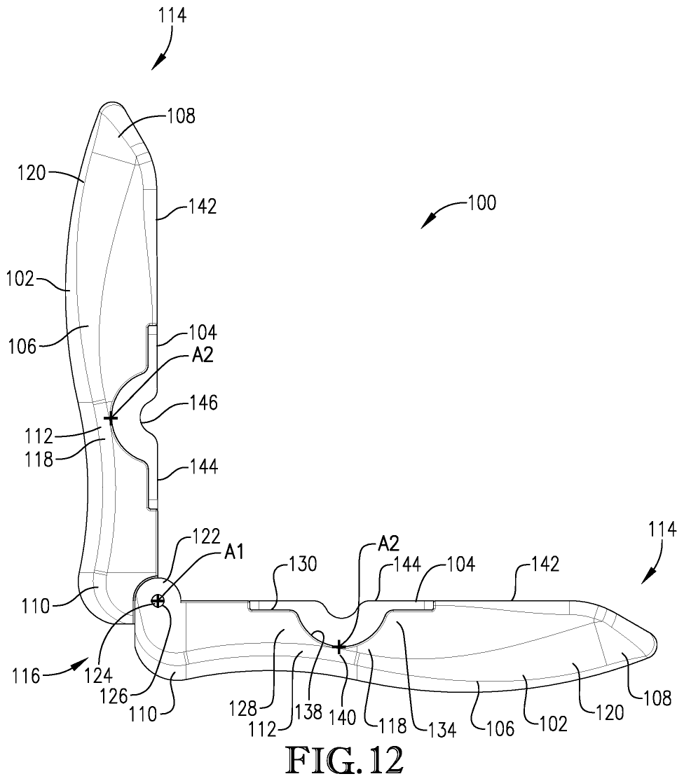

In the depicted embodiment, tool levers 102 are pivotally attached relative to one another at the pivot joint 126 (see FIGS. 9 and 10). Specifically, distal ends 116 of the illustrated tool levers 102 are pivotally attached to each other at a pinned joint that permits the tool levers 102 to swing relative to each other about a lateral pivot axis A1 (see FIG. 12). In particular, the tool levers 102 include pivot connectors 122 that are intercalated with one another and joined by a pin element 124 so that the tool levers 102 are swingably attached to each other (see FIGS. 9 and 10). However, the pivot joint for swingably connecting the tool levers may be cooperatively formed by alternative pivot connectors. An alternative pivot joint may also include a structure other than the pin element (e.g., a threaded fastener) for joining the tool connectors. In alternative embodiments, the pivot connectors may be attached to one another without a pin structure. Also, the pivot joint may be provided by a living hinge. The tool levers 102 are relatively swingable into and out of a closed position to grip the catheter hub H (or another part of the catheter).

Tool handles 120 of the tool levers 102 extend adjacent the proximal lever ends 114 and are configured to be grasped by a user to swing the tool levers 102 between the closed position and an open position (see FIG. 10). The tool handles 120 preferably present opposed stop surfaces 142 that engage one another in the closed position to restrict the gripping force applied by the jaws 118.

Each flexible gripping element 104 is supported by and cooperates with the respective lever body 106 to form a corresponding jaw 118 of the gripping tool 100. The jaws 118 are opposed and swingable toward and away from each other. Jaws 118 of the gripping tool 100 cooperatively engage the catheter hub H (or another part of the catheter) in the closed position. As will be explained, the jaw frame 128 of the lever body 106 is configured to resiliently flex the gripping element 104 when the gripping tool 100 is closed. The gripping element 104 has a unitary form and presents an exposed gripping surface 144. Each gripping surface 144 defines a lateral channel 146 to receive part of the catheter (see FIG. 9).

The illustrated gripping element 104 preferably comprises a unitary flexible insert that is configured to be inserted into the socket 132. The insert is preferably attached within the socket 132 (e.g., where the gripping element is adhered or molded onto the lever body). However, the gripping element 104 may be otherwise attached to the lever body 106. For example, an alternative gripping element may be removably attached to the lever body (e.g., with one or more fasteners).

Gripping element 104 comprises a resilient gripping element material that is relatively more flexible than a material of the respective lever body 106. The gripping element 104 is operable to resiliently flex for conforming with part of the catheter hub (or other catheter element) and for restricting the gripping force applied by the jaws 118 when the catheter hub is held by the jaws 118 in the closed position.

In preferred embodiments, the gripping element material of gripping element 104 comprises a synthetic resin material, such as an elastomeric material. For instance, the gripping element material may include one or more polymer materials, such as a low density polyethylene (LDPE), polypropylene, and/or other polymers. Preferably, the gripping element material has a Shore A durometer that ranges from about 70 to about 80 to ensure proper flexibility and ability to grip.

It is within the scope of the present invention for at least one of the gripping elements to be alternatively configured (e.g., for being supported by the lever body). For instance, at least one of the gripping elements may be alternatively shaped for removable engagement with the lever body. It will also be understood that the gripping surface may be alternatively shaped for alternative gripping engagement with a structure (e.g., a catheter element) to be grasped by the gripping tool.

As noted above, an alternative gripping element may be fixed to the lever body. For instance, the gripping element and lever body may be formed of different materials but fixed relative to each other (e.g., where a relatively flexible gripping element is molded or adhered to a relatively rigid lever body).

As noted above, the resilient hinge 112 of each tool lever 102 permits relative flexing between the proximal and distal lever sections 108,110. In particular, the resilient hinge 112 joins the lever sections 108,110 and permits relative flexing about a lateral flexing axis A2 (see FIG. 12) to restrict the clamping force applied by the jaws 118 when the gripping tool 100 is in the closed position. In the depicted embodiment, the jaw frame 128 and the gripping element 104 cooperatively provide the resilient hinge 112.

Each resilient hinge 112 is configured to be shifted between a relaxed condition (see FIG. 9) and a flexed condition (not shown). When shifting from the relaxed condition to the flexed condition, the proximal lever sections 108 of the tool levers 102 are pivoted about the hinges 112 toward each other. When shifting the depicted lever sections 108 toward the flexed condition, the end walls 136 and side walls 134 of the jaw 118 are pivoted about the corresponding hinge 112 toward each other. When shifting from the flexed condition to the relaxed condition, the proximal lever sections of the tool levers 102 are pivoted about the hinges 112 away from each other.

Again, the lever body includes a jaw frame 128 that extends at least partly around the gripping element 104. In the illustrated embodiment, the resilient hinge 112 includes the living hinge 140, which is provided by the jaw frame 128 (see FIG. 10). More particularly, the end walls 136 and side walls 134 of the jaw frame 128 cooperatively define the living hinge 140.

The flexibility associated with the lever body 106, particularly the living hinge 140 of the jaw frame 128, permits the tool lever 102 to bend slightly to accommodate the clamped catheter hub, while reaching a “hard stop point” when the stop surfaces 142 adjacent the proximal ends 114 come into contact with each other. As a result, the flexible lever body 106 is configured to restrict the clamping force applied by the jaws 118 when the gripping tool 100 is in the closed position. The living hinge 140 preferably urges the tool lever 102 to return from the flexed condition toward the relaxed condition.

Again, the gripping element 104 cooperates with the jaw frame 128 to provide the resilient hinge 112. The end walls 136 of the jaw frame 128 support and engage the gripping element 104. As the tool lever 102 is shifted toward the flexed condition, the end walls 136 cooperatively resiliently flex the gripping element 104 so that the gripping element 104 provides a spring of the resilient hinge 112. Preferably, the flexed gripping element 104 urges the tool lever 102 to return from the flexed condition toward the relaxed condition. Thus, in the illustrated embodiment, the gripping element 104 and jaw frame 128 cooperate to urge the tool lever 102 out of the flexed condition toward the relaxed condition.

Additional advantages of the various embodiments of the invention will be apparent to those skilled in the art upon review of the disclosure herein and the working examples below. It will be appreciated that the various embodiments described herein are not necessarily mutually exclusive unless otherwise indicated herein. For example, a feature described or depicted in one embodiment may also be included in other embodiments, but is not necessarily included. Thus, the present invention encompasses a variety of combinations and/or integrations of the specific embodiments described herein.

As used herein, the phrase “and/or,” when used in a list of two or more items, means that any one of the listed items can be employed by itself or any combination of two or more of the listed items can be employed. For example, if a composition is described as containing or excluding components A, B, and/or C, the composition can contain or exclude A alone; B alone; C alone; A and B in combination; A and C in combination; B and C in combination; or A, B, and C in combination.

The present description also uses numerical ranges to quantify certain parameters relating to various embodiments of the invention. It should be understood that when numerical ranges are provided, such ranges are to be construed as providing literal support for claim limitations that only recite the lower value of the range as well as claim limitations that only recite the upper value of the range. For example, a disclosed numerical range of about 10 to about 100 provides literal support for a claim reciting “greater than about 10” (with no upper bounds) and a claim reciting “less than about 100” (with no lower bounds).

Although the above description presents features of preferred embodiments of the present invention, other preferred embodiments may also be created in keeping with the principles of the invention. Such other preferred embodiments may, for instance, be provided with features drawn from one or more of the embodiments described above. Yet further, such other preferred embodiments may include features from multiple embodiments described above, particularly where such features are compatible for use together despite having been presented independently as part of separate embodiments in the above description.

The preferred forms of the invention described above are to be used as illustration only, and should not be utilized in a limiting sense in interpreting the scope of the present invention. Obvious modifications to the exemplary embodiments, as hereinabove set forth, could be readily made by those skilled in the art without departing from the spirit of the present invention.

The inventor hereby states his intent to rely on the Doctrine of Equivalents to determine and assess the reasonably fair scope of the present invention as pertains to any apparatus not materially departing from but outside the literal scope of the invention as set forth in the following claims.

1. A medical adapter gripping tool operable to removably hold a catheter element, said medical adapter gripping tool comprising:
a pair of elongated tool levers each including a longitudinally extending lever body and presenting opposite lever ends,
said tool levers being pivotally attached relative to one another at a pivot joint that permits the tool levers to swing relative to each other about a lateral pivot axis, with the tool levers being relatively swingable into and out of a closed position to grip the catheter element,
each of said tool levers defining a jaw located between the lever ends,
said jaws being opposed and swingable toward and away from each other, with the jaws cooperatively engaging the catheter element in the closed position,
a respective tool lever including a gripping element supported by and cooperating with the respective lever body to form a corresponding jaw, with the gripping element presenting an exposed gripping surface,
said gripping element comprising a resilient gripping element material, with the gripping element operable to resiliently flex for conforming with part of the catheter element and restricting the gripping force applied by the jaws when the catheter element is held by the jaws in the closed position.

2. The medical adapter gripping tool as claimed in claim 1,
said respective lever body including an outer jaw margin that extends at least partly around the gripping element.

3. The medical adapter gripping tool as claimed in claim 1,
at least one of said tool levers including proximal and distal lever sections and a resilient hinge that joins the lever sections and permits relative flexing between the lever sections about a lateral flexing axis to restrict the gripping force applied by the jaws in the closed position.

4. The medical adapter gripping tool as claimed in claim 3,
said respective lever body including an outer jaw margin that extends at least partly around the gripping element,
said outer jaw margin of the respective lever body at least partly defining the resilient hinge.

5. The medical adapter gripping tool as claimed in claim 4,
said resilient hinge including a living hinge at least partly provided by the outer jaw margin.

6. The medical adapter gripping tool as claimed in claim 4,
said gripping element being integrally formed with the outer jaw margin.

7. The medical adapter gripping tool as claimed in claim 1,
said tool levers including opposed flexible gripping elements, which include the first-mentioned gripping element,
each of said gripping elements supported by and cooperating with the respective lever body to form a corresponding jaw, with each gripping element presenting an exposed gripping surface.

8. The medical adapter gripping tool as claimed in claim 1,
each of said tool levers including a tool handle,
said lever ends of each tool lever comprising proximal and distal lever ends, with the tool handles extending adjacent the respective proximal lever ends and the pivot joint located adjacent to the distal lever ends.

9. The medical adapter gripping tool as claimed in claim 1,
said tool handles each including a stop element, with the stop elements engaging one another in the closed position to restrict the gripping force applied by the jaws.

10. The medical adapter gripping tool as claimed in claim 1, further comprising:
a spring interconnecting the tool levers and providing the pivot joint, with the spring urging the tool levers out of the closed position.

11. The medical adapter gripping tool as claimed in claim 10,
said spring comprising a torsion spring with a pair of prongs, with each prong being attached to a respective one of the tool levers.

12. The medical adapter gripping tool as claimed in claim 11,
said tool levers each presenting a slotted opening, with the prongs of the torsion spring being inserted into respective slotted openings.

13. A medical adapter gripping tool operable to removably hold a catheter element, said medical adapter gripping tool comprising:
a pair of elongated tool levers each including a longitudinally extending lever body and presenting opposite lever ends,
said tool levers being pivotally attached relative to one another at a pivot joint that permits the tool levers to swing relative to each other about a lateral pivot axis, with the tool levers being relatively swingable into and out of a closed position to grip the catheter element,
each of said tool levers defining a jaw located between the lever ends,
said jaws being opposed and swingable toward and away from each other, with the jaws cooperatively engaging the catheter element in the closed position,
at least one of said tool levers including proximal and distal lever sections and a resilient hinge that joins the lever sections and permits relative flexing between the lever sections about a lateral flexing axis to restrict the force applied by the jaws when the catheter element is held by the jaws in the closed position.

14. The medical adapter gripping tool as claimed in claim 13,
said respective lever body including a jaw frame that at least partly defines the resilient hinge.

15. The medical adapter gripping tool as claimed in claim 14,
said resilient hinge including a living hinge provided by the jaw frame.

16. The medical adapter gripping tool as claimed in claim 13,
each of said tool levers including the proximal and distal lever sections and the resilient hinge.

17. The medical adapter gripping tool as claimed in claim 13,
each of said tool levers including a tool handle,
said lever ends of each tool lever comprising proximal and distal lever ends, with the tool handles extending adjacent the respective proximal lever ends and the pivot joint located adjacent to the distal lever ends.

18. The medical adapter gripping tool as claimed in claim 17,
said tool handles each including a stop element, with the stop elements engaging one another in the closed position to restrict the gripping force applied by the jaws.

19. The medical adapter gripping tool as claimed in claim 13, further comprising:
a spring interconnecting the tool levers and providing the pivot joint, with the spring urging the tool levers out of the closed position.

20. The medical adapter gripping tool as claimed in claim 19,
said spring comprising a torsion spring with a pair of prongs, with each prong being attached to a respective one of the tool levers.

21. The medical adapter gripping tool as claimed in claim 20,
said tool levers each presenting a slotted opening, with the prongs of the torsion spring being inserted into respective slotted openings.

22. A method of removably gripping a catheter element with a medical adapter gripping tool, said method comprising the steps of:
(a) having the catheter element positioned between the jaws of pivotally-attached tool levers while the tool levers are opened; and
(b) having the tool levers shifted relative to each other to close the jaws against the catheter element, with a gripping element of at least one jaw resiliently flexing to conform with part of the catheter element and restrict the gripping force applied by the jaws when the catheter element is held by the jaws in the closed position.

23. The method as claimed in claim 22, further comprising the step of:
(c) having at least one of the tool levers flex at a resilient hinge that joins proximal and distal lever sections and permits relative flexing between the lever sections about a lateral flexing axis to restrict the force applied by the jaws when the catheter element is held by the jaws in the closed position.
